(19) United States
(12) Patent Application Publication
(10) Pub. No.: US 2009/0306533 A1
(43) Pub. Date: Dec. 10, 2009

(54) Stroke Inducing and Monitoring System and Method for Using the Same

(76) Inventors: Patrick J. Rousche, Downers Grove, IL (US); Terry C. Chiganos, JR., Downers Grove, IL (US); Winnie Jensen, Aalorg (DK)

Correspondence Address: LICATA & TYRRELL P.C., 66 E. MAIN STREET, MARLTON, NJ, 08053, US

(21) Appl. No.: 12/162,140

(22) PCT Filed: Jan. 24, 2007
(86) PCT No.: PCT/US07/60965

§ 371 Date: Nov. 12, 2008

Related U.S. Application Data
(60) Provisional application No. 60/763,102, filed on Jan. 26, 2006.

(51) Int. Cl. A61B 5/0476; A61M 31/00

(52) U.S. Cl. 600/544; 604/500

(57) ABSTRACT

The present invention is a system and method for realtime monitoring of neural responses to stroke. The system of the present invention provides a component for inducing a localized stroke and one or more sensors for monitoring molecular and cellular physiological events before, during and after the stroke. Methods for inducing a stroke, monitoring neural responses, and identifying neuroprotective strategies and/or agents with a model are also provided.

This application claims benefit of U.S. Provisional Patent Application Ser. No. 60/763,102, filed Jan. 26, 2006, the contents of which are incorporated herein by reference in their entirety.

This invention was made in the course of research sponsored by the National Science Foundation (Grant No. BES-0233529). The U.S. government has certain rights in this invention.

### BACKGROUND OF THE INVENTION

Stroke is a sudden loss of brain function resulting from interference with the blood supply to the central nervous system leading to cerebral ischemia. Although the pathophysiologic mechanisms may vary, stroke often leads to permanent neurological deficit. Unmitigated cerebral ischemia secondary to reduced cerebral blood flow (CBF) gives rise to a variety of motor, sensory and cognitive deficits depending on the location and nature of the cerebrovascular event (Dobkin (2005) N. Engl. J. Med. 352:1677-84; Dobkin (2004) Ann. NY Acad. Sci. 1038:148-70). The extent of brain injury depends on several factors including the duration of blood flow reduction and the anatomical distribution of the damaged vessels. The likelihood of clinical improvement after stroke is directly attributable to the extent of the hypoxia-induced damage (Dobkin (2005) supra). No fully curative treatment exists for patients with neurological deficits resulting from neural tissue loss. Cortical neurons exhibit different types of cell death depending on the specific characteristics of the cell and the nature of the ischemia (Lipton (1999) Physiol. Rev. 79:1431-568). Unlike certain epithelial tissues, the cerebral cortex has only limited capability of replacing large populations of damaged cells following hypoxic injury (Gu, et al. (2000) J. Cereb. Blood Flow Metab. 20:1166-73). While specific regions such as the subventricular zone (SVZ) and subgranular zone (SGZ) retain some capacity for neurogenesis via endogenous precursor cells (Arvidsson, et al. (2002)Nat. Med. 8:963-70), the primary mechanism of functional recovery is considered to be a property of the redistribution of existing cortical representations among surviving (and typically neighboring) neural tissue. Reorganization of axonal connections between surviving neurons proximal to the infarct as well as interhemispheric projections has been implicated in the partial recovery of lost function (Carmichael (2003) Neuroscientist 9:64-75; Kijkhuizen, et al. (2001) Proc. Natl. Acad. Sci. USA 98:12766-7). Presumably, such axonal reorganization and/or redistribution of cortical representations will influence the electrophysiological properties of the associated neurons.

Despite this general knowledge regarding reorganization, there is little specific information available regarding the actual dynamic electrophysiological responses of neurons before, during and after a stroke. In related work, the effects of asphyxial cardiac arrest on somatosensory thalamo-cortical relays has been described (Muthuswamy, et al. (2002) Neuroscience 115:917-29). This study tracked the dissociative effects of global brain injury on somatosensory processing. The detrimental effects of global ischemic injury, however, will undoubtedly differ from the dynamic electrophysiological profile of neuron clusters after a focal cortical deficit. The modulation of multi-unit neural electrical activity after transient middle-cerebral artery occlusion has also been investigated and compared to overt sensorimotor deficits. This study showed that the suppression of multi-unit activity is highly correlated with the degree of sensorimotor dysfunction (Moyanova, et al. (2003) J. Neurol. Sci. 212:59-67).

Analysis of local reorganization (or plasticity) after focal infarction has shown that neurons within the infarct zone and surrounding cortex will exhibit demonstrable changes in function as part of the natural stroke response (Buchkremer-Ratzman, et al. (1996) Stroke 27:1105-9; Fujioka, et al. (2004) Stroke 35:e346-8; Neumann-Haefelin and Witte (2000) J. Cereb. Blood Flow Metab. 20:45-52; Witte, et al. (2000) J. Cereb. Blood Flow Metab. 20:1149-65). The brain areas surrounding the infarct core as well as corresponding contralateral regions exhibit sustained excitability changes shortly after infarction as measured using low resolution evoked potentials (Buchkremer-Ratzman, et al. (1996) supra; Fujioka, et al. (2004) supra). Within the acute phase (<24 hours after infarction), periinfarct regions exhibit hyperexcitability; a restorative process hypothesized to enhance the effects of peripheral stimuli on damaged neurons (Fujioka, et al. (2004) supra). Other studies report decreased excitability of periinfarct tissue 7 days post-infarction (middle cerebral artery occlusion) (Neumann-Haefelin and Witte (2000) supra). In this case, hypoexcitability is likely to be the summative result of neuron density loss, functional suppression of individual neurons or inflammatory reactions (Neumann-Haefelin and Witte (2000) supra). To clearly elucidate the dynamic electrophysiological responses associated with cortical infarction, a system capable of the continuous observation of neuron-specific electrical properties before, during and after a stroke is needed. If the degree of inter-patient variability regarding the electrical state of the post-ischemic cortex can be completely determined, patient-specific treatment protocols can be better tailored to reverse damaging excitability changes as they occur.

Devices for monitoring neuronal activity have been suggested. For example, U.S. Pat. No. 6,263,225 discloses a dual purpose multicontact electrode assembly capable of monitoring and inactivating neurons. The apparatus is an electrode support shaft having a distal end and a proximal end, wherein a plurality of neuron-monitoring microelectrodes are positioned along the distal end of the electrode support shaft, and each one of a plurality of lesion-producing macroelectrodes are placed adjacent to each one of the plurality of microelectrodes.

U.S. Pat. No. 7,010,356 teaches a multichannel electrode for recording, stimulating and lesioning a target site as well as providing imaging, drug delivery and therapeutic capabilities. Electrode channels of the device are micromachined or microlithographically etched into an electrically conductive backbone, wherein each set of channels performs a specific function such as recording or stimulating and/or lesioning.

U.S. Pat. No. 6,526,309 discloses an optical system and method for transcranial in vivo examination of brain tissue including a spectrophotometer coupled to an array of optical fibers and a processor.

Further, U.S. Pat. No. 6,277,082 teaches a device for detecting ischemia in tissue, by temporarily altering the temperature of the tissue and then monitoring the tissue's thermal response as it returns to normal body temperature.

Similarly, U.S. Pat. No. 6,697,657 teaches laser-induced fluorescence attenuation spectroscopy for the detection of ischemia and hypoxia in biological tissue.

Moreover, while devices have been suggested for inducing or removing arterial occlusions, these devices do not provide for simultaneous real-time analysis of the dynamic responses of neurons proximate to the occluded vessel. See, e.g., U.S. Pat. Nos. 6,120,499; 6,379,325; 6,942,657; and 6,022,309 and U.S. patent application Ser. No. 09/727,603.

Thus, there is a need in the art for a system which allows real-time evaluation of neural responses before, during, and subsequent to a controlled ischemic challenge. The present invention meets this need in the art.

### SUMMARY OF THE INVENTION

The present invention is a system for real-time monitoring of neural responses to stroke. The system is composed of at least one sensor and a guide, which is proximate to said sensor and adapted for receiving a stroke-inducing component so that upon the induction of a stroke, neural response to the stroke can be monitored via the sensor. In one embodiment, the system further includes a stroke-inducing component. In other embodiments, the system is implanted into at least one region of the brain of a mammal to provide a model for monitoring neural responses and identifying neuroprotective agents. Methods for inducing a stroke in the brain of a mammal and using the model for real-time monitoring of neural responses and identifying neuroprotective agents are also provided.

### BRIEF DESCRIPTION OF THE DRAWINGS

FIG. 1 depicts a system for simultaneously inducing and monitoring a stroke.

FIG. 2 depicts a system for inducing a stroke via photothrombosis with simultaneous biochemical, chemical and/or electrical neural monitoring via a plurality of implantable sensors.

FIG. 3 is a sectional view depicting configurations of the guide 40 and sensors 30. In FIG. 3A, sensors 30 are configured radially around guide 40. In FIG. 3B, sensors 30 are adjacent to guide 40. In FIG. 3C, sensors 30 are adjacent to and in-line with guide 40. In FIG. 3D, microwire sensors 30a, microdialysis sensors 30b, and carbon fiber sensors 30c are bundled and configured radially around guide 40.

FIG. 4 are graphs showing the analysis of the auditory response after the onset of infarction. FIG. 4A shows the average normalized peak firing rate (PFR) and cumulative activity (CA) for data from eight animals. FIG. 4B shows an exemplary PFR for an abrupt decrease profile. The PFR profile was classified as abrupt if there existed a continuous decrease that accounted for ≧90% of the overall loss (i.e., all clusters). The shaded area indicates the continuous decrease used for linear regression analysis for the cluster. FIG. 4C shows an exemplary PFR for a gradual decrease profile. For gradual decrease profiles, all data points were used to generate the linear regression model. FIG. 4D shows the average normalized peak firing rate for abrupt and gradual clusters with the linear regression models for each curve. *, gradual linear regression, m=−9.930±2.240 (×10⁻⁴). #, abrupt linear regression, m=−39.99±4.801 (×10⁻⁴). The linear approximation for abrupt neuron clusters only used data points during the linear decline (300-540 seconds).

FIGS. 5A and 5B are photomicrographs showing the infarct border and peri-lesional region (Nissl stain) 28 days after photothrombosis. FIG. 5A shows clear delineation between the dense, heavily stained normal cortex and the sparsely populated penumbra region with leukocyte infiltration visible. FIG. 5B shows the peri-lesional region with blood vessel, wherein the magnified capillary (in box on left) is encapsulated with inflammatory cells.

FIG. 6 shows a peri-stimulus time histograms (PSTH).

FIG. 7 shows the relative blood perfusion during control conditions (FIG. 7A), euthanasia (FIG. 7B) and stroke (lesion core) (FIG. 7C).

FIG. 8 shows the average perfusion change at the infarct core relative to the initial value for control (n=5), euthanasia (n=6) and focal infarction (n=7) groups. The asterisks above the core and euthanasia samples indicate statistical distinction from control (t-test, α<0.05). The errors bars indicate the standard deviation of each sample.

FIG. 9 shows box plots of peak (FIG. 9A) and mean (FIG. 9B) firing rates at 1 hour after infarction.

### DETAILED DESCRIPTION OF THE INVENTION

A stroke occurs when blood flow to an area of the brain is interrupted. There are generally two types of stroke, ischemic stroke (e.g., thrombotic stroke and lacunar infarction of small arterial vessels) and stroke resulting from the breakage or blowout of a blood vessel in the brain, i.e., hemorrhagic stroke. As with ischemic stroke, hemorrhagic stroke destroys brain cells. However, hemorrhagic stroke also poses other complications as well, including increased pressure on the brain or spasms in the blood vessels, both of which endanger the patient.

Temporal characterization of the dynamic molecular and physiological responses to stroke provides information about local neuronal plasticity and cortical reorganization. Furthermore, a continuous neural response signature following prolonged hypoxia allows for an assessment of the neuroprotective capacity of treatments designed to combat secondary mechanisms of damage and/or augment the natural response.

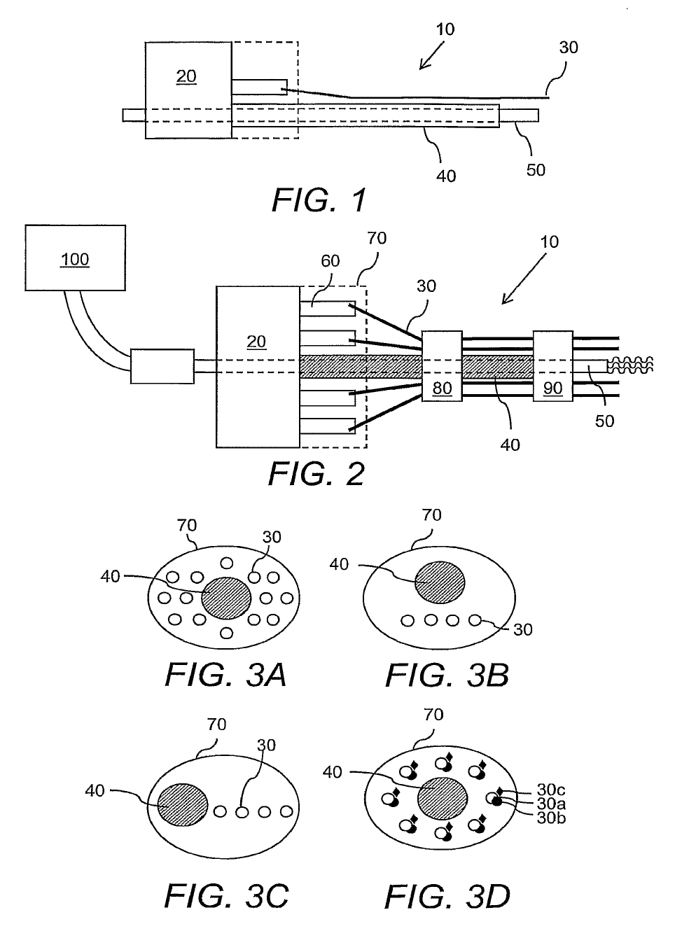

A novel system and method have now been developed for real-time monitoring of dynamic biochemical, chemical, and/or electrical neural responses before, during and subsequent to a controlled ischemic challenge at specific locations relative to the lesion border. Referring to FIG. 1, the present system 10 is composed of base 20 having attached thereto at least one implantable neural sensor 30 and a guide 40, which is adapted for receiving stroke-inducing component 50. In particular embodiments, system 10 employs a plurality of sensors 30 (FIG. 2). When employing a plurality of sensors, said sensors 30 are proximate to, but independent of, guide 40 and can be configured, e.g., radially around (FIG. 3A), adjacent to guide 40 (FIG. 3B), adjacent to and in-line with guide 40 (FIG. 3C), or any other pattern or configuration. When in use, system 10 is implanted into a desired region of the brain; stroke-inducing component 50 is introduced through guide 40 and activated to generate a localized or focal stroke at the desired brain region; and biochemical, chemical, and/or electrical neural responses before, during and subsequent to the ischemic challenge are detected and monitored with sensor 30.

As the stroke-inducing component can be retracted after inducing a stroke, the stroke-inducing component can be removed from the brain, the exposed brain tissue can be sealed off from the outside environment and monitoring of the recovery process can be carried out over an extended period of time (e.g., days, weeks, or months) via the neural sensors. Given the independence of the stroke-inducing component from the sensor(s), the sensor(s) can be implanted in the same region of the brain as the guide for the stroke-inducing component; or alternatively, the sensor(s) and the guide can be configured so that they are located in adjacent regions of the brain. Moreover, in certain configurations, a plurality or array of sensors can be implanted at neurons located at different depths in the brain.

The guide for the stroke-inducing component can be made of any suitable material and can take any shape depending on the stroke-inducing component employed. Desirably, the guide is biocompatible and capable of being sterilized. Likewise, a variety of suitable stroke-inducing components can be used in accordance with the present invention, wherein the component is selected based upon the type of stroke to be monitored. In one embodiment, an ischemic stroke is induced. In accordance with this embodiment, blood vessel occlusion is achieved using electromagnetic radiation. For example, radio frequency electrical energy in the range of 0.3 to about 1.5 megahertz is known for use in occluding blood vessels. See U.S. Pat. No. 6,120,499. In particular embodiments, laser or visible light (e.g., 300 to 700 nm) is used in combination with a photosensitizing agent to induce a focal infarction. Activation of a light source initiates a photochemical cascade ultimately resulting in the formation of free-radical oxygen species, which initiate a cascade of intravascular biomolecular events leading to microvascular platelet aggregation and disruption of the blood-brain barrier. This process, commonly referred to as photothrombosis creates reproducible, physiologically relevant lesions with precise control of location, diameter and depth (Watson et al., (1985) Ann. Neurol. 17:497-504; Ginsberg and Busto (1989) Stroke 20:1627-42; Hu, et al. (1999) Brain Res. 849:175-86). Photothrombosis employs intravenous injection of a photosensitizing agent (e.g., Rose bengal, a fluorinated derivative of fluorescein) and exposing a selected area of tissue to light to induce clotting. Although the mechanism of microvasculature occlusion is artificial, the ensuing tissue damage is morphologically consistent with naturally occurring ischemic infarcts (Witte, et al. (2000) supra). Photothrombotic insult generates an ischemic penumbra that can expand for up to 24 hours following illumination (Lee, et al. (1996) Stroke 27:2110-9), rendering the periinfarct tissue amenable to neuroprotective intervention (Webster, et al. (1995) Stroke 26:444-50). Induction of blood vessel inclusions by photothrombosis is well-known and described in U.S. Pat. No. 5,053,006, incorporated herein by reference in its entirety.

In another embodiment, a hemorrhagic stroke is induced. In accordance with this embodiment, the flow of blood is disrupted by breaking blood vessels via ultrasonic mechanisms, laser (e.g., holmium laser), or combinations thereof. See, e.g., WO 2004/052181 which discloses ultrashort laser pulses of lower energy for controllably producing hemorrhage or thrombosis.

The stroke-inducing component can be composed of any suitable material which transmits the desired energy. For example, fibers (i.e., fiber optics), glass, quartz, or polymeric materials suitably conduct light energy in the form of visible, ultraviolet light, infrared radiation, or coherent light, e.g., laser light. Selection of an appropriate material for the required wavelength is well within the skill of one in the art.

To detect and monitor neural events that occur during and after an ischemic event, the sensor(s) of the present invention is implanted below the pia mater (i.e., intracranial) so that direct contact with one or more individual neurons and/or the surrounding extracellular fluid is achieved. For example, the sensor(s) of the present invention can be implanted into any region of the cerebral cortex including the primary motor cortex, supplementary motor cortex somatosensory cortex, visual cortex, auditory cortex, Wernicke's area, Broca's area, or other cortical or intracranial regions of the brain. The sensor(s) of the present invention is used to acutely or chronically monitor any number of neural responses including molecular and physiological parameters such as electrical signals in response to external stimuli, oxygen, glucose, pH, amino acids, protein biomarkers and the like. The instant system can have one sensor or a plurality of sensors (e.g., 2, 3, 4, 5, G, 7, 8, 9, 10 or more). Moreover, the instant system can have one sensor that detects one parameter (e.g., electrical activity), one sensor that detects multiple parameters (e.g., electrical activity and oxygen level), or multiple sensors that detect multiple parameters. To detect multiple parameters of one individual neuron, the sensors can be bundled or braided. While the sensors of the present invention can take on any shape or configuration, sensors of the instant invention are generally wires or tubes having a diameter in the range of 5 to 200 micrometers, or more desirably in the range of 10 to 100 micrometers. Moreover, sensors can be stiff or flexible, to “flow” with each pulsation of the brain tissue thereby avoiding disturbances in the surrounding tissues during extended periods of monitoring. Sensors suitable for use in accordance with the instant system are well-known in the art.

For example, an enzyme-based micron-scale sensor is disclosed in U.S. Pat. No. 6,802,957 for detecting glucose, glutamate, lactate or hydrogen peroxide. Similarly, U.S. Pat. No. 6,576,102 discloses analyte sensors which can be adapted for use in accordance with the instant system. Ischemia and hypoxia are both conditions that deprive tissue of oxygen, leading to anaerobic metabolism and the accumulation of the metabolic coenzyme NADH. Therefore, monitoring concentrations of NADH can also be used to indirectly monitor oxygen levels. Moreover, ion-selective electrodes are useful for measuring levels and small changes in ion, neurotransmitter and hormone concentrations in and near cells. Suitable electrodes of this type are commercially available (e.g., Molecular Devices Corporation, Sunnyvale, Calif.). Microdialysis sensors for neurotransmitter and amino acid detection, among other compounds, are available commercially (e.g., CM Microdialysis, Solna, Sweden) Moreover, carbon fiber amperometry is also embraced by the present invention for sensing and monitoring ions and biomolecules (Koh (2006) Methods Mol. Biol. 337:139-53). As indicated, it is contemplated that sensors of the invention can be bundled to detect multiple parameters at one location (e.g., one neuron). By way of illustration, FIG. 3D shows bundling of microwire electrode sensors 30a, microdialysis sensors 30b, and carbon fiber sensors 30c to detect multiple parameters at one location, wherein the bundles are configured around guide 40.

While all cells maintain an electrical potential across their membranes, neurons are highly specialized in using membrane potentials (action potentials) to transmit signals from one part of the body to another. The action potential of a neuron represents a transient depolarization of its membrane over a period of a few milliseconds. Action potentials, in turn, have proved to be valuable indicators of the physiological status and functionality of those neurons. Accordingly, particular embodiments embrace a system wherein at least one sensor is capable of detecting neural electrical activity in response to stimuli.

Electrophysiological effects of stroke can be monitored using a variety of electroconductors including, but not limited to microwire electrodes, silicon-based electrodes and the like. These types of electrodes are known to provide reliable measurements without delivering compromising damage to the brain (Prechtl, et al. (2000) Proc. Natl. Acad. Sci. USA 97(2):877-882). When employing a microwire electrode; desirably the electrode has an impedance suitable for recording action potentials from individual cells (e.g., between 100 ohms and 2-3 Mohms). Single contact microwire electrodes can be employed as well as microelectrodes containing a pair of contacts (corresponding to a bipolar contact) in close juxtaposition. Moreover, each microelectrode can be a tripolar contact array (i.e., stereotrode; McNaughton, et al. (1983) J. Neurosci. Methods 8:391-397). As the skilled artisan will appreciate, other configurations are also possible.

As demonstrated herein, the functional response of the infarct core and periinfarct zone can be assessed by creating PSTH versus Time (PSTHvT) plots. Neurons in the peri-lesional zone outside the infarct core demonstrate significant functional changes as a result of acute local plasticity, but the time scale of those changes is far greater compared to neurons within the infarct core.

Signal from electrodes of the instant system can be amplified at, or adjacent to, the point of contact with the neuron or amplified extracranially. For example, when employing a microelectrode containing a pair of contacts, a differential amplifier (Bak Electronics, Germantown, Md.), and differential recordings can be made from one contact relative to the other. Using the system of the present invention, current flow as well as mean firing rates of neurons can be monitored over time to assess the electrical response properties of neurons as a result of acute plasticity/reorganization of the post-infarct cortex.

Signals from sensors of the present invention can be passed through discriminatory circuits to insure that only waveforms with specific characteristics are counted as the activity from one neuron. Moreover, the instant system can be attached to a processor and/or readout device such as a personal computer to convert, display, and/or manipulate measured parameters obtained by the sensor(s).

In particular embodiments, the system of the present invention is implanted into one or more regions of the brain of a mammal, e.g., a rat, pig, mouse, dog, cat, cow, goat, chicken, and the like, to provide a model for acute and chronic monitoring of neural responses to stroke and identifying neuroprotective agents. Speech and language problems arise when brain damage occurs in the language centers of the brain. Due to the brain's ability to learn and change (i.e., plasticity and reorganization), other areas can adapt to take over some of the lost functions. Accordingly, not only does the instant model provide a means for analyzing excitotoxicity and reperfusion injury to identify targets for prevention and treatment of brain damage from stroke, the instant model also allows the skilled artisan to monitor the recovery process.

The system and model of the present invention find application in methods for monitoring neural responses to stroke, identifying and evaluating neuroprotective agents and strategies. A method for monitoring neural responses to stroke involves implanting into one or more regions of the brain of a mammal a system of the present invention, inducing a stroke via the stoke-inducing component of the system, and detecting neural responses to the stroke via one or more sensors. By detecting and monitoring neural responses to stroke, particularly for extended periods of time (e.g., days, weeks, or months), the acute mechanisms of damage to the infarct core and surrounding cortex can be characterized as can post-stroke reorganization. With this characterization, cellular, molecular, genetic or the like, targets can be identified to prevent or minimize damage as well as speed the recovery process.

In accordance with a method for identifying neuroprotective agents, the system of the present invention is implanted into one or more regions of the brain of a mammal, the animal is administered (e.g., orally, intravenously, transdermally, etc.) a test agent, a stroke is induced, and biochemical, chemical, and/or electrical neural responses to the stroke are detected and measured. Any improvement in biochemical, chemical, or electrical neural responses (e.g., an increase in peak firing rate, an increase in PSTH response degradation time, increase in oxygen levels, decrease in anabolic processes and the like) when compared to a control (e.g., a mammal subjected to a stroke without receiving the test agent) indicates that the test agent provided neuroprotection. In addition to administering the test agent before the stroke, the test agent can also be administered subsequent to the stroke to identify agents that accelerate or facilitate the recovery process.

Test agents which can be screened in accordance with the instant method include any number of small molecule antioxidants, antioxidant enzymes, natural or synthetically produced molecules, plant extracts, as well as strategies such as electrical stimulation, novel physical therapy routines, and the like.

As exemplified herein, the system and method of the invention allow for excellent reproducibility and precise lesion volume and location as well as long-term observation of neural activity in nearby brain regions. For example, the data disclosed demonstrate a clear and consistent effect of hypoxia on the evoked electrical activity of neurons located within an infarct core.

Using the instant system, one of skill in the art has the unique ability to control the location of cortical recordings relative to the lesion border with recordings obtainable throughout the evolution of the stroke. For example, using the instant system it is now possible to monitor the extinction patterns of neurons located at the core of a stroke as well as neurons surrounding the core that will ‘take over’ the function of the dying neurons and lead to stroke recovery. A specific understanding of neural response profiles will provide therapies or medications designed to augment the natural response and promote constructive reorganization of surviving neurons.

The invention is described in greater detail by the following non-limiting examples.

### Example 1

### Single Sensor System and Analysis of the Auditory Cortex

Electrode Manufacture. Continuous electrical monitoring was performed using a single microwire electrode implanted to a sub-pial depth of 800 μm in the rat primary auditory cortex (A1). The electrode was hand-fabricated from inexpensive materials using an adaptation from the art (Williams, et al. (1999) Brain Res. Protocols 4:303-13). A 2-cm length of 100 μm (outside diameter), tungsten microwire insulated with TEFLON™ (polytetrafluoroethylene; A-M Systems Inc.®, Carlsborg, Wash.) was soldered to a connector and insulated with an epoxy shell to mechanically stabilize the solder connection (two-part quick-dry epoxy; RadioShack Inc.®, Fort Worth, Tex.) (see FIG. 1). Before cortical implantation, the electrode tip was cleaned in 70% isopropyl alcohol and the assembly was gas sterilized with ethylene oxide to remove particulate matter from the electrode surface.

Surgical Procedure. Prior to surgery, male Sprague-Dawley (SD) rats (350-500 grams, n=8; Taconic Inc., Hudson, N.Y.) received a bolus intramuscular injection of ketamine (100 mg kg⁻¹), xylazine (5 mg kg⁻¹) and acepromazine (2.5 mg kg⁻¹) (KXA) for induction of anesthesia. Supplemental doses of KXA mixture were used as needed to maintain a surgical plane of anesthesia for the duration of the experiment. The pulse rate, oxygen saturation and paw-pinch reflex were used to assure a consistent depth of anesthesia.

A 2-cm incision above the midline cranial suture provided access to the skull surface. Prior to exposure of the implant target, a bone screw was placed over the contralateral hemisphere posterior to bregma and anterior to the lambdoid suture to serve as a local ground for differential recording. A craniectomy was performed on the lateral aspect of the cranium posterior to the lateral suture to expose the dura above the primary auditory cortex (−3.3 mm to −6.3 mm anterior-posterior and 6 mm lateral relative to bregma). The specific site of implantation was identified using stereotactic coordinates, bony landmarks and surface blood vessel patterns. Following excision of the dura, the microwire was lowered into the brain using a micromanipulator until the pia was visibly punctured (typically less than 2 mm). After puncture, the microwire was retracted to a maximum depth of 800 μm beneath the cortical surface. The placement of the electrode in primary auditory cortex was verified by detecting short-latency (10-25 ms) stimulus-evoked signals from the site of implantation.

Assessment of Electrical Activity. A PC-controlled Tucker-Davis Technologies (TDT; Alachua, Fla.) System 3 data acquisition system with real-time digital signal processing was used to record the electrical signals from the cortex and generate the auditory stimulus for characterization of neuronal function. The implanted electrode was connected to a custom headstage (unity gain, high impedance input) and preamplifier. The signal was digitized at 25 kHz using a 16-bit analog-to-digital converter (ADC) (±7 mV operating range, 6 mV RMS noise floor, 0.2 μV resolution) before being multiplexed along a fiber-optic cable to the TDT processor bank. Using custom software, the raw signal was filtered (800-8000 Hz) and an automatic action potential detection threshold was set to a multiple of the background noise (typically 1.5 times the time-averaged baseline amplitude without stimulus presentation).

To elicit firing from the primary auditory neurons, a 250 μs free-field, contralateral auditory click stimulus was presented at 2 Hz (120 presentations minute⁻¹) from a loudspeaker located at 1.5 meters from the animal. The software recorded the timestamps of each signal that exceeded threshold as well as the timestamps of the stimulus presentation.

The timestamps of the spikes recorded for each neuron cluster allow for the analysis of cortical function. The peri-stimulus time histogram (PSTH) is a modified cross correlation between action potential timestamps and stimulus timestamps that is used to quantify the functional electrical output of neurons in vivo. A single PSTH can be used to establish several quantitative parameters of electrophysiological function including relevant density (RD) which is the sum of all statistically significant bin counts for a single PSTH or PSTH versus time normalized to the pre-infarct average; peak firing rate (PFR), the maximum post-stimulus firing rate observed over all bins for a single PSTH; cumulative activity (CA), which is the sum of the firing rates over all bins for a single PSTH and is a measure of the total electrical activity of a given neuron cluster; and response onset latency (ROL), which is the first bin after stimulus presentation (time=0 ms) when a statistically significant firing rate is observed. Each bin that exceeds the 95% confidence limit assuming an independent Poisson distribution of spontaneous firing is considered statistically significant (Abeles (1982) J. Neurosci. Methods 5:317-25).

In the present study, a dynamic profile of neural function was obtained by evaluating the PSTH versus time (PSTHvT) following infarction. The PSTHvT is a compilation of several, distinct PSTHs created using a moving time window during continuous presentation of the auditory stimulus. To generate the PSTHvT, data from a graph showing peak firing rate are color-coded for firing rate and plotted as a single column. Each column represents the color-coded PSTH for all auditory stimuli (120 clicks) delivered during the next consecutive minute, etc. To obtain adequate time resolution for discerning changes after infarction, the PSTHvTs were created by using overlapping 1-minute time windows (thus 120 stimulus events contribute to each column) shifted forward in time by 30 seconds.

Induction of Focal Infarction. Focal infarct was created using a modified photothrombosis procedure known in the art (Watson, et al. (1985) supra). Prior to the craniectomy and electrode insertion, a microcatheter (0.762 mm outside diameter; SAI Inc.) was inserted into the femoral vein for later delivery of the rose bengal (RB) dye. The catheter was filled with saline to reduce the likelihood of thrombus formation during prolonged hemostasis. A fiber-optic light probe (Intralux® 6000; Volpi Inc., Auburn, N.Y.) with heat filter (Ealing Inc., Rocklin, Calif.) was lowered to approximately 1 mm from the cortical surface such that the implanted electrode was located within the beam illumination pattern. The electrode was approximately located in the center of the incident light beam, assuring complete microvascular occlusion surrounding the microwire. As the brain surface was illuminated, an RB dye solution (10 mg ml⁻¹, 0.9% saline solution, 2 mg/100 mg body weight) was injected at 1.0 ml minute⁻¹. Illumination continued for 20 minutes following the RD infusion. For the remainder of the study, initiation of photothrombosis was defined as the onset of RB infusion. Following successful catheterization and illumination, the area of cortex subject to illumination always appeared blanched compared to the surrounding brain, providing immediate visual confirmation of a local perfusion deficit. To assess the infarction method, 5 μm coronal sections from one animal were Nissl stained 14 days after initiation of infarction for morphological assessment of the local tissue.

Electrophysiological Evaluation of Normal Auditory Cortex. Primary auditory cortex was chosen for the present study due to the relative ease by which the dynamic function of primary auditory neurons is quantified using standard electrophysiological techniques. In this study, a broadly activating free-field click stimulus was chosen for its ability to easily and consistently induce neural activity in primary auditory cortex. Furthermore, primary auditory responses show no extended sign of instability or stimulus adaptation over the recording period. However, more complex (and neuron-specific) auditory stimuli such as pure tones with frequency and/or amplitude modulation and generation of spectral-temporal receptive fields could also be employed. It is contemplated that target-specific auditory stimuli coupled with measurement of tissue oxygenation levels can provide the most information regarding induced change in neural function. Ultimately, it is this functional change that should be minimized and/or controlled if clinical outcome is to be improved. Because motor cortex infarction typically results in the most debilitating clinical deficits (often interrupting language capabilities and vital activities of daily living), it remains an excellent target for this type of evaluation.

Before infarction, baseline recordings were performed for up to 1 hour to verify response stationarity. A PSTH composed from >120 stimulus presentations was generated along with the corresponding raster plot. The bin size was 1 ms and 3 bin Gaussian smoothing was applied. The PSTH plot revealed a peak firing rate (PFR) and response onset latency (ROL) of 518.40 spikes s⁻¹ and 13.0 ms, respectively (multi-unit recording). In a typical analysis, the standard deviation of the peak firing rate was only 2.9% of the mean (mean=495.52 spikes s⁻¹, standard deviation=14.52 spikes s⁻¹). Furthermore, for each PSTH, the first bin to achieve statistical significance (ROL) occurred 13 ms after stimulus presentation. The relatively small standard deviation of the PFR over time and the constancy of the ROL substantiate the stability of the multi-unit response prior to ischemic insult.

In another control recording from a different animal, a PSTHvT was created for stimuli presented for up to 1 hour. For this case, auditory stimuli were presented in 5-minute epochs, with 5 minutes of silence between each stimulus block. With no local infarction, auditory neural responses remained relatively stable, particularly with respect to ROL (mean ROL=12.97±0.18 ms) and PFR (mean PFR=592.5±15.18 ms). Furthermore, the PSTHvT did not indicate accommodation of the auditory responses, i.e., no observed diminution of stimulus-evoked firing patterns in response to the prolonged, repetitive stimulus.

Electrophysiological Evaluation of Infarct Core. Using a single microwire during photo-initiated cortical infarction, PSTHvTs showed neural activity (normalized to peak firing above background) measured 2 minutes before infusion of the rose bengal followed by continuous recording for another 13 minutes during concurrent cortical surface illumination. The general trends exhibited a clear and consistent extinction of auditory-driven neural responses. To quantify the response loss, the loss of relevant density for each neuron was calculated and used to identify the time to response extinction. To eliminate the variability of pre-infarct firing rates between experiments, the relevant density was normalized to the pre-infarct level, thereby establishing a dimensionless quantitative measure of total significant activity for the entire recording session. The time to complete response extinction (TRE) was defined as the first PSTH within the contiguous sequence with no relevant density (no bins above the 95% confidence interval). The average TRE (n=8) was 439±92 seconds following initiation of photothrombosis, and the average RD (n=8) for the entire recording session was 12.34±2.9. Although the time-course of response extinction varied within the infarct core (as evidenced by the relatively large standard deviation), complete loss of response was seen for all neuron clusters within 600 seconds. Despite the complex nature of the system and the numerous physiological parameters that could influence the acute response to ischemia, the TRE and RD exhibited remarkable consistency between animals, substantiating the reproducibility of the disclosed method.

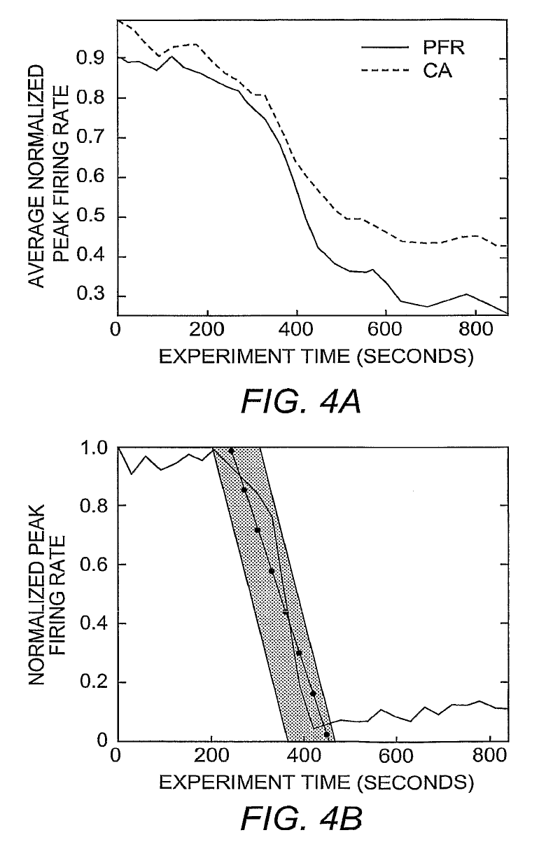

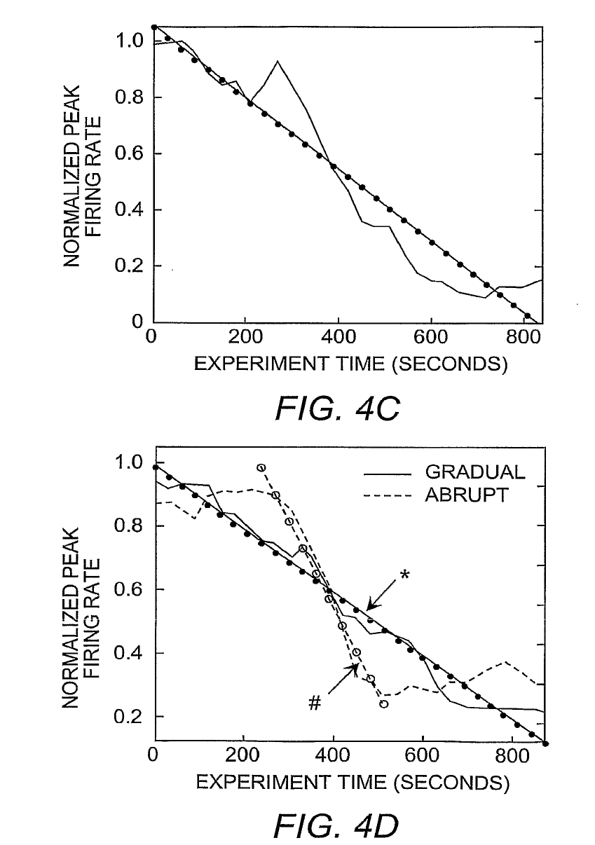

FIG. 4A depicts the averaged PFR and cumulative activity (CA) curves for all eight neuron clusters. In addition to the normalized peak response, the cumulative activity provides an additional measure of overall excitability. Both the averaged PFR and CA curves approached background levels within the 15-minute recording session. Background activity was defined as the observed electrical activity when no external stimulus was applied. To further analyze the unique response of the infarct core, the PSTHvTs were grouped according to the temporal degradation profile of the PFR. The decrease of the peak firing rate after infarction was empirically classified as gradual or abrupt. The PFR profile was considered abrupt if a continuous, decreasing segment of the normalized PFR curve existed that accounted for ≧90% of the total peak firing loss. The linear regression model for clusters classified as abrupt was obtained by considering only the continuous decrease (FIG. 4B). If no such segment existed, the profile was classified as gradual and all points of the normalized PFR were included for linear regression analysis (FIG. 4C). Linear regression analysis of the PFR curves for the gradual and abrupt clusters revealed a mean slope for abrupt clusters more than four times greater than the mean slope for the gradual clusters. FIG. 4D shows the averaged PFR for gradual and abrupt clusters with the linear regression model for both curves. The empirical classification scheme separated the EP response according to the decay profile of the peak firing rate. The rate of decay of the PFR exhibited significant variability between animals as evidenced by the disparate slopes, indicating a unique, individual response for each neuron cluster.

The variability of the temporal degradation of the peak firing rate (abrupt versus gradual) may have been due to unique electrophysiology, or the consequence of physiologic and/or anatomical factors. The loss of stimulus-evoked firing after infarction is linked to cortical tissue oxygenation levels. The oxygen level for each neuron cluster depends on tissue perfusion and the relative anatomical distribution of the local microvasculature. Variations of baseline oxygen saturation and core body temperature may affect the observed EP profile. Furthermore, the time interval between dye injection and capillary occlusion is dependent on the circulation time of the dye, which is linked to the cardiovascular dynamics of the rat (e.g., pulse rate, stroke volume, total blood volume, mean arterial pressure, etc). The observed variability of the time to response extinction (300-600 second post-infusion), however, was relatively small considering possible confounding factors. Overall, the present system demonstrated consistency and reproducibility, both necessary factors for the quantitative analysis of neuron function.

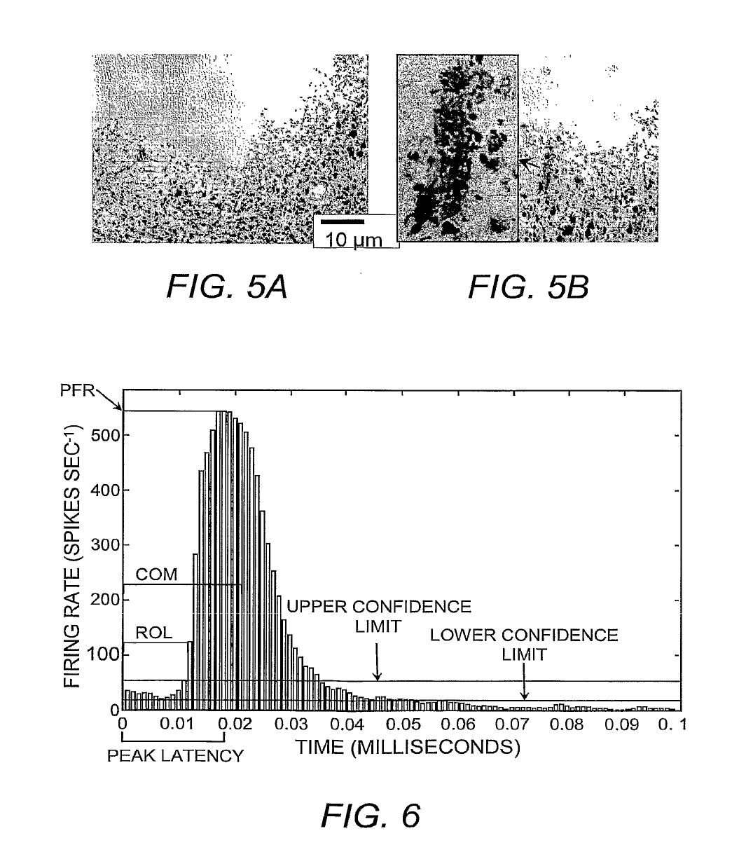

Morphological Assessment of Infarction Zone. The photothrombosis-mediated perfusion deficit resulted in prolonged hypoxia and eventual neuron death according to histological assessment. The cortical infarcts were easily recognizable on the surface of the cortex after surgery, and the mottled appearance of the large cortical blood vessels within the lesion was consistent with clot formation. Following injection and light exposure, a circular zone of pallor consistent with the diameter of the fiber-optic probe (˜2 mm) was observed on the cortical surface, indicating decreased tissue perfusion. The coronal histological sections demonstrated that the photothrombosis procedure caused cell death and remodeling of the local cellular architecture of the cortex. At 4 weeks post-infarction, the Nissl-stained tissue exhibited classic wedge-shaped architecture with a consistent periinfarct or penumbra region containing inflammatory cell infiltration and loss of cell density (FIG. 5A). Microvessels within the penumbra were encased in inflammatory cells indicating disruption of the blood-brain barrier during the natural recovery attempt (FIG. 5B).

### Example 2

### Multi-Sensor System

A multi-sensor system was also generated and used to monitor the electrophysiological effects of photothrombosis. Referring to FIG. 2, the exemplary system 10 contained four Tungsten microwire sensors 30 with a cylindrical guide tube 40 for insertion of a fiber optic or laser light probe as the stroke-inducing component 50. In detail, a four pin connector 60 (MOLEX®, Inc., Lisle, Ill.) was sealed at the base 20 using a thin layer of dental acrylic (polymethyl methacrylate, PMMA) before Tungsten microwire sensors 30 insulated with TEFLON™ (polytetrafluoroethylene, 100 μm total diameter) were soldered to each pin connector 60. In addition to microwire sensors 30, a cylindrical plastic guide 40 (2 mm inner diameter) was attached to base 20 to allow for the insertion of the fiber optic light probe 50. The system 10, from base 20 to the end of electrode wire sensors 30 was about one inch. The impedance of each connection was tested before the connector 60 was encapsulated by an epoxy shell 70. The protruding microwire sensors 30 along with the guide tube 40 were passed through an alignment dye 80 and a second application of PMMA was used to affix the microwire sensors 30 in the intended configuration. A final island of PMMA 90 was applied for stability. When in use, the small diameter fiber optic probe 50 for inducing photothrombotic induction of stroke was passed through the guide 40 after the microwire sensors 30 were implanted into the brain. The system 10, therefore, ensures that the light source 100 will illuminate a section of cortex adjacent to the implanted microwire sensors 30.

### Example 3

### Comparative Electrophysiology within the Core and Peri-Lesional Regions after Focal Ischemic Stroke

Cortical hypoxia secondary to cerebrovascular occlusion produces an ischemic lesion with two functionally distinct regions. An understanding of the electrophysiological (EP) profile of neuron clusters within the infarct core and those in the outer penumbra region better defines the therapeutic window for the acute management of stroke. To compare the continuous electrical signals from core (n=8) and peri-lesional (n=8) neuron clusters, microwire electrodes were acutely implanted into the primary auditory cortex of 16 anaesthetized rats. Neural activity was recorded before, during and after an induced focal infarction. The dynamic EP response was correlated with laser-Doppler blood perfusion measurements within the ischemic core and peri-lesional zone.

To quantify the EP response during the hyper-acute injury phase, Tungsten microwire electrode channels located at 0.5, 1.0, 1.5, and 2.0 mm from the edge of the focal lesion (see configuration depicted in FIG. 3C) were implanted into the primary auditory cortex of male, Sprague Dawley rats (n=16). Focal infarction was induced using a photochemical method to ensure precise lesion location and volume. The photosensitive dye rose bengal was infused via an indwelling femoral vein catheter. As the dye circulated through the cerebral vasculature, concurrent external illumination (fiber optic light probe, 1.5 mm outside diameter) initiated microvascular coagulation limited to the cylindrical zone of illumination.

Analysis of auditory cortex function was carried out. An example of a Gaussian smoothened (3-bin) peri-stimulus time histogram (PSTH) is shown in FIG. 6. As indicated herein, the PSTH bins the timestamps of the action potentials from each neuron cluster relative to the presentation of the auditory stimulus. The PSTH provides a quantitative assessment of neuron function for comparison before and after infarction. The PSTH over time is used to identify the dynamic profile of the functional response over time. A peri-stimulus time histogram versus time (PSTHvT) is a compilation of several distinct PSTHs creating using smaller moving time windows. The PSTHvT, therefore, provides an assessment of EP changes during the acute recovery window.

Laser-Doppler blood perfusion measurements were also carried out. Low-intensity laser light was reflected off moving red blood cells within a specific tissue volume. The Doppler shift was extracted from the reflected light to determine the relative amount of tissue perfusion.

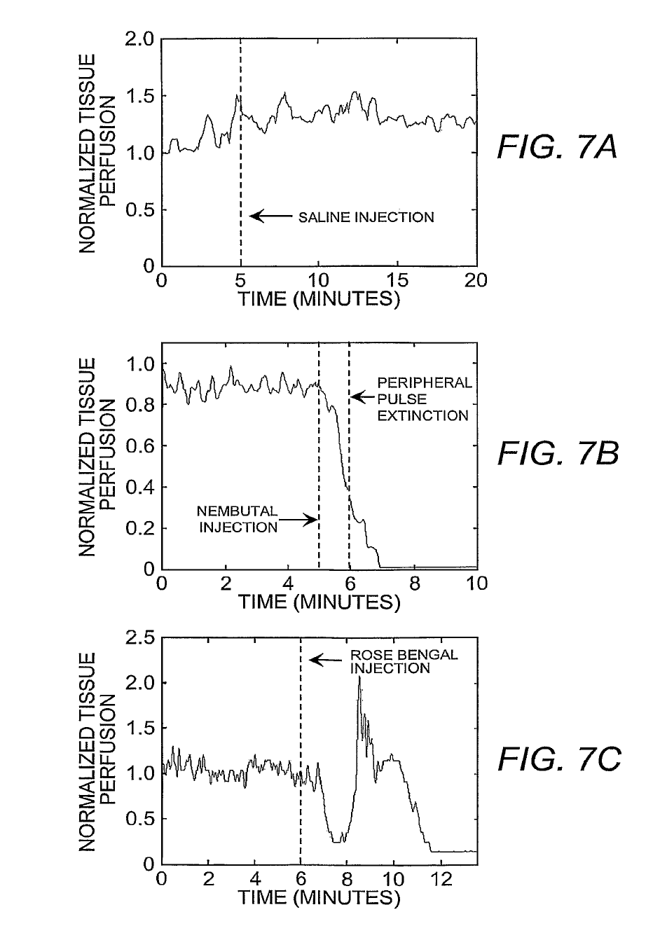

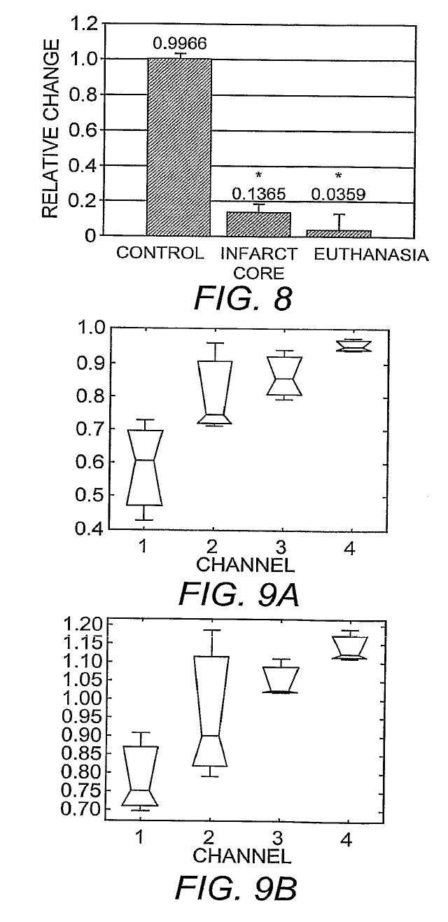

The infarct core analysis (n=8) indicated an average TRE of 439+/−92 seconds and an average relevant density of 12.34+/−2.9. Furthermore, within the zone of illumination, the EP response was consistently lost within 10 minutes of rose bengal injection. As shown in FIG. 7, the relative perfusion dramatically decreased during both euthanasia (FIG. 7B) and focal infarction (FIG. 7C). Average perfusion change relative to the initial value for control (n=5), euthanasia (n=6) and focal infarction (n=7) groups is shown in FIG. 8. When the probe was kept at a fixed distance from the cortex during control experiments, the level of perfusion remained virtually constant. The flow dropped to ˜14% and ˜4% of original values after focal infarction and euthanasia, respectively.

Peri-lesional recordings indicated a progressive degradation of the peak and mean firing rates, along with a steady increase of response latency. Specifically, the average PFR decrease for all channels was 71.2%, whereas ROL increased by a factor of 1.71+/−0.28 and PL increased by a factor of 1.3+/−0.25. ANOVA revealed a distance-dependent influence on the peak (FIG. 9A) and mean (FIG. 9B) firing rates, but no effect on latency factors or center of mass.

### Example 4

### Intracortical Motor Cortex Responses to Ischemic Stroke

To demonstrate motor cortex responses to ischemic stroke, 5 male Sprague-Dawley rats were analyzed using the system disclosed herein. A craniectomy was performed over the area related to forelimb movement. Each rat had a 16-channel microwire array (100 μm wire diameter) implanted into their M1 or primary motor cortex. Channels 1-4 of the array were located 3.0 mm from the edge of the focal lesion, whereas channels 5-8, channels 9-12, and channels 13-16 of the array were respectively located 2.5 mm, 2.0 mm, and 1.5 mm from the edge of the focal lesion. A cuff electrode was implanted around the Ulnar nerve and electrical stimulation (pulse width=100 μs, frequency ˜2 Hz) was provided to evoke antidromic, cortical M1 responses. An ischemic infarct was created by light activation of Rose Bengal (1.3 mg/100 mg body weight) and occlusion of blood vessels was easily determined. Histological evaluation verified a change in cell density and occurrence of inflammatory cells. Data were colleted up to 7 hours after induction of the ischemic infarct. Peri-stimulus time histograms (PSTHs) were synchronized to the onset of the ulnar nerve stimulation. The mean PSTH activity and onset latency (i.e., the time where the PSTH curve cross the 95% confidence interval, upper confidence interval as shown in FIG. 6) was calculated. PSTH versus time plots were generated.

The results of this analysis indicated that the activity of the channels closest to the ischemic core experienced the largest decrease. Hyperexcitability was seen in a majority of the channels, followed by a gradual decrease in the cortical activity over time. The onset and degree of hyperexcitability was dependent on the distance of the channel from the ischemic core. Moreover, the onset latency increased in the majority of the channels. Before the onset of the ischemia the latency was 20.1±4.6 ms (mean±std across all 4 animals) and this increased to 30.0±9 ms.

What is claimed is:

1. A system for real-time monitoring of neural responses to stroke comprising at least one sensor and a guide, which is proximate to said sensor and adapted for receiving a stroke-inducing component, such that upon the induction of a stroke, neural response to the stroke can be monitored via the sensor.

2. The system of claim 1, further comprising a stroke-inducing component.

3. A model for real-time monitoring of neural responses to stroke comprising a mammal having implanted into at least one region of the brain the system of claim 1.

4. A model for identifying neuroprotective agents comprising a mammal having implanted into at least one region of the brain the system of claim 1.

5. A method for inducing a stoke in the brain of a mammal comprising implanting into at least one region of the brain of a mammal the system of claim 1; introducing a stroke-inducing component through the guide; and activating the stroke-inducing component thereby inducing a stroke in the brain of the mammal.

6. A method for real-time monitoring of neural responses to stroke comprising implanting into at least one region of the brain of a mammal the system of claim 2; inducing a stroke via the stroke-inducing component; and detecting neural responses with the sensor so that neural responses to the stroke are monitored.

7. A method for identifying a neuroprotective agent comprising implanting into at least one region of the brain of a mammal the system of claim 2; inducing a stroke via the stroke-inducing component; administering a test agent to the mammal before or after the stroke; and detecting bio-chemical-electrical neural responses with the sensor, wherein modulation of the neural responses in the presence of the test agent as compared to a control is indicative of a neuroprotective agent.
